(19) United States
(12) Patent Application Publication
(10) Pub. No.: US 2005/0178388 A1
(43) Pub. Date: Aug. 18, 2005

(54) Throat mask with soft tube

(76) Inventor: Chi Chao Kuo, Taichung Hsien (TW)

Correspondence Address: Chi Chao Kuo, 235 Chung - Ho, Box 8-24, Taipei, TW

(21) Appl. No.: 10/780,228

(22) Filed: Feb. 18, 2004

(51) Int. Cl. A62B 9/06; A61M 16/00

(52) U.S. Cl. 128/207.14

(57) ABSTRACT

The throat mask comprises a breath tube having an end portion which is expanded as a throat sheet with a shape like a water drop; a resisting sheet enclosing the throat sheet; a front end of the resisting ring being extended with a guide unit which is a deformable semi-elliptical soft tube with two ends; the two ends of the soft tube being connected to the resisting ring; an airbag enclosing a periphery of the resisting ring so as to avoid the resisting ring and the guide sheet to contact the throat directly. The soft tube has a soft structure deformable to fit the space receiving the guide unit. After the guide unit passes into the throat, the soft tube can collapse downwards or swings at two sides so as to make the throat feel easy and the guide unit will not hinder the throat.

### FIELD OF THE INVENTION

The present invention relates to throat masks, and particularly to a throat mask with a soft tube to fit the space receiving the guide unit; after the guide unit passes into the throat. The soft tube can collapse downwards or swings between two sides so as to make the throat feel easy and the guide unit will not hinder the throat.

### BACKGROUND OF THE INVENTION

The throat mask of the present invention is mainly used to assist breath in surgery or assist eating of patient after surgery. In general, in surgery, the doctor must insert a throat mask to the patient so that the patient can breathe or eat easily. In general, the throat mask has a solid guide unit 13A, see FIG. 1. It is un-deformable so that it cannot deform with the shape of the patient throat. This will make the patient feel uneasy. In some cases, a front end of the guide unit 13A will bend so as to make the breathing action uneasy to induce an emergency accident.

### SUMMARY OF THE INVENTION

Accordingly, the primary object of the present invention is to provide a throat mask. The throat mask comprises a breath tube having an end portion which is expand as a throat sheet with a shape like a water drop; a resisting sheet enclosing the throat sheet; a front end of the resisting ring being extended with a guide unit; the guide unit being a deformable semi-elliptical soft tube with two ends; two ends of the soft tube being connected to the resisting ring so that the guide unit having a hollow structure; an airbag enclosing a periphery of the resisting ring so as to avoid the resisting ring and the guide sheet to contact the throat directly. The soft tube has a soft structure which is deformable to fit the space receiving the guide unit. When the tip of the soft tube resists against the periphery of the throat, the soft tube will collapse downwards or swings at two sides so as to make the throat feel easy and the guide unit will not hinder the throat.

The various objects and advantages of the present invention will be more readily understood from the following detailed description when read in conjunction with the appended drawing.

### BRIEF DESCRIPTION OF THE DRAWINGS

FIG. 1 is a perspective view of the prior art throat mask.

FIG. 2 is a perspective view of the throat mask of the present invention.

FIG. 3A is a schematic view showing that the guide unit of the present invention is reduced inwards.

FIG. 3B is a schematic view showing that the guide unit of the present invention swings between two ends.

FIG. 4 shows one use of throat mask of the present invention.

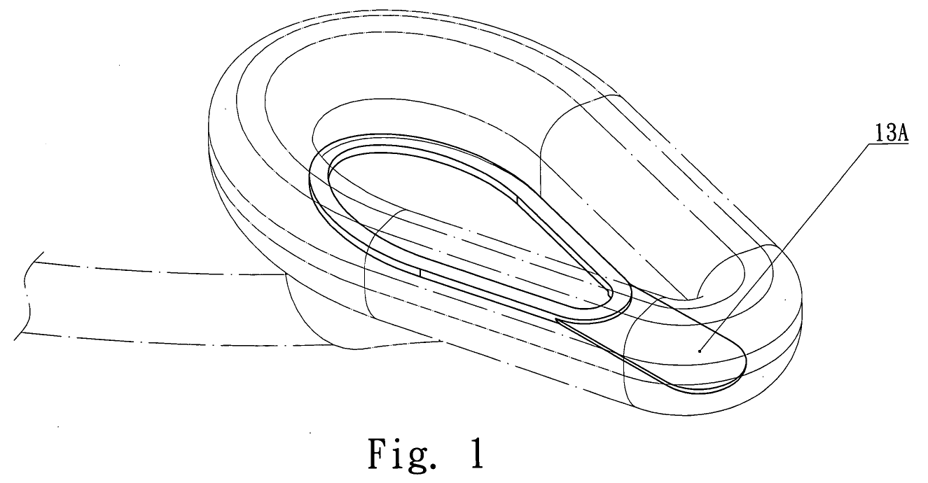

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

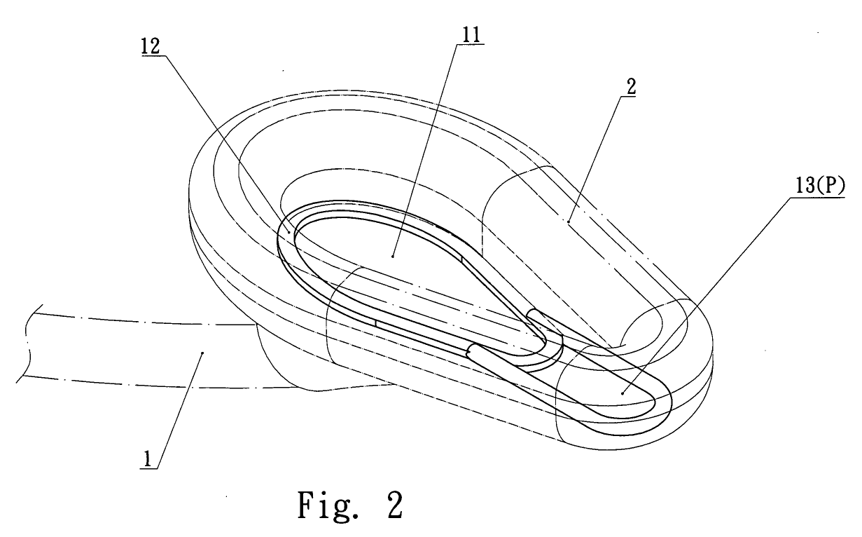

With reference to FIG. 2, the structure of the present invention is illustrated. The throat mask of the present invention includes the following elements.

A breath tube 1 has an end portion which is expand as a throat sheet 11 with a shape like a water drop. A resisting sheet 12 encloses the throat sheet 11. A front end of the resisting ring 12 is extended with a guide unit 13. In the present invention, the guide unit 13 is a semi-elliptical soft tube P with two ends. Two ends of the soft tube P are connected to the resisting ring 12 so that the guide unit 13 has a hollow structure.

An airbag 2 encloses a periphery of the resisting ring 12 so as to avoid the resisting ring 12 or the guide sheet 13 to contact the throat directly.

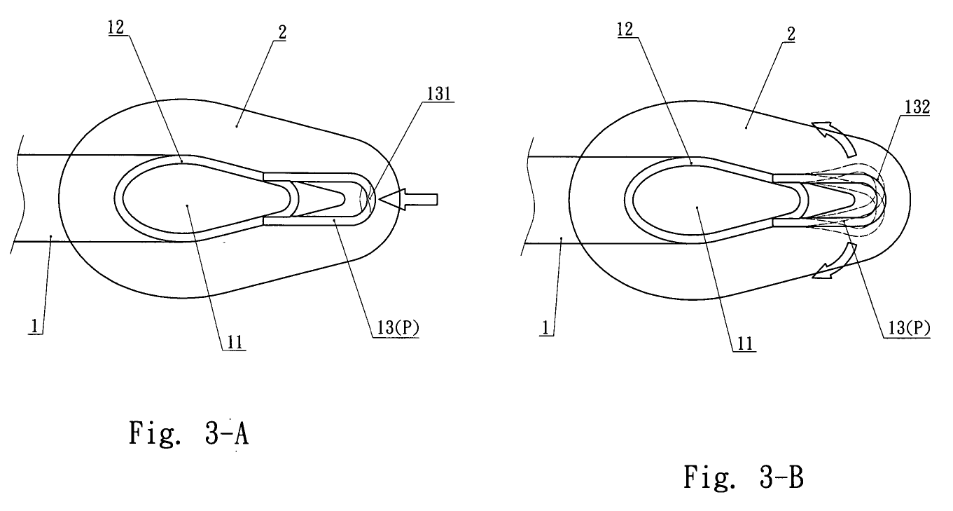

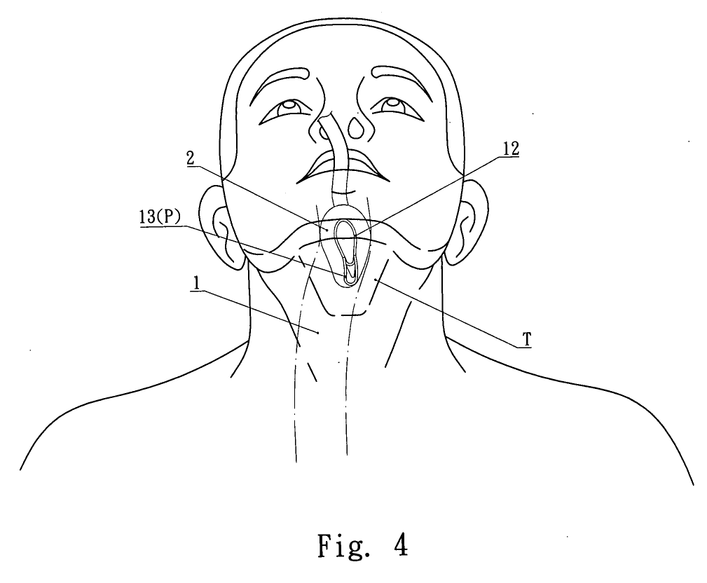

Thereby in the present invention, since the soft tube P has a soft structure which is deformable to fit the space receiving the guide unit 13. After the guide unit 13 passes into the throat T, when the tip of the soft tube P resists against the periphery of the throat, the soft tube P will collapse downwards (referring to FIG. 3A) or swings between two sides (referring to FIG. 3B) so as to make the throat feel easy and the guide unit 13 will not hinder the throat.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A throat mask comprising:
a breath tube having an end portion which is expand as a throat sheet with a shape like a water drop; a resisting sheet enclosing the throat sheet; a front end of the resisting ring being extended with a guide unit; the guide unit being a deformable semi-elliptical soft tube with two ends; the two ends of the soft tube being connected to the resisting ring so that the guide unit having a hollow structure;
an airbag enclosing a periphery of the resisting ring so as to avoid the resisting ring and the guide sheet to contact the throat directly;
wherein the soft tube has a soft structure which is deformable to fit the space receiving the guide unit; after the guide unit passes into the throat, when the tip of the soft tube resists against the periphery of the throat, the soft tube will collapse downwards or swings to make the throat feel easy and the guide unit will not hinder the throat.
